(19) United States
(12) Patent Application Publication
(10) Pub. No.: US 2005/0021084 A1
(43) Pub. Date: Jan. 27, 2005

(54) Bone treatment device and method

(76) Inventors: William Weijia Lu, Hong Kong (CN); Keith Dip Kei Luk, Hong Kong (CN); John Chi-Yan Leong, Hong Kong (CN); Shew Ping Chow, Hong Kong (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/843,580

(22) Filed: May 12, 2004

Related U.S. Application Data
(60) Provisional application No. 60/471,348, filed on May 19, 2003.

(51) Int. Cl. A61B 17/08

(52) U.S. Cl. 606/218

(57) ABSTRACT

The present invention can provide a bone treatment device, which allows for reinforced security at the bone interface and/or for delivery of a bone treatment material into a bone portion. The bone treatment device can have a shaft member with first and second end portions. A delivery channel can be formed in the shaft member to assist administering a bone treatment material to a predetermined bone portion or tissue. The delivery channel can have a chamber formed in the shaft member and extending from the first end portion toward the second end portion. The delivery channel can also have one or more port members formed on the shaft member and communicating with the chamber.

### CROSS-REFERENCE TO RELATED PATENT APPLICATION

This patent application claims benefit to U.S. Provisional Patent Application No. 60/471,348 filed May 19, 2003.

### FIELD OF THE INVENTION

The present invention relates generally to a bone treatment device, such as a bone screw. In particular, the present invention relates to a bone fixation device having an interior chamber and one or more port members that allow for the delivery of a bone treatment material from the interior chamber into a bone portion. Additionally, the present invention relates to a bone treatment method.

### BACKGROUND OF THE INVENTION

Bone screws have been applied in osteopathy, such as for the treatment of bone fractures, osteoporotic factures, or bone deformations and as a standard anchoring technique. For example, bone screws, such as pedicle screws, can be used as an anchoring system to secure an implant, such as a spinal implant, to a bone. There are two types of screws commonly available on the market. One type is the self-tapping screw, which can be directly inserted into bone. The other type is the non-self-tapping screw which requires a pre-drilled hole at a pre-determined site before inserting the screw into a bone. The diameter of the pre-drilled hole is slightly smaller than the diameter of the non-self-tapping screw.

When a bone screw is inserted into a healthy bone, a bonding force between the screw and the bone is created to prevent the implant from pulling-out or loosening after fixation. For example, in spinal trauma operations, fractured vertebrae are usually stabilized by temporarily implanting spinal instrumentation. Subsequently, with the use of bone grafting techniques, the fractured vertebrae can be permanently fused-up over a period of time up to two years.

When bone screws are used in a patient having osteoporosis, the bonding force between the screw and the bone can be weak, which can result in loosening or easy pulling-out of the bone screw from the bone interface. For example, a fractured vertebrae cannot be stabilized by conventional spinal implants due to the poor bonding force at the interface of the screw and the bone, such as bone screw to femur head and neck. When using bone cement to fill up the osteoporotic vertebrae through holes drilled in the vertebrae prior to bone screw insertion, the amount of cement and the area filled by the cement are difficult to control. In addition, cement leakages, which frequently happen, can cause complications.

U.S. Pat. No. 6,368,319 to Schaefer discloses a pedicle screw having a safety mechanism at the screw's end to prevent the screw from being pulled out of the bone. The safety mechanism in Schaefer is a lock or washer that is threaded onto the end of the screw and secures the screw to the bone. U.S. Pat. No. 6,375,657 to Doubler et al. discloses a screw having a threading. The screw in Doubler et al. has a tapered core and fixed pitch threads that have a compound curved interior and constant width crest. U.S. Pat. No. 6,183,472 to Lutz discloses a pedicle screw and assembly aid. The screw in Lutz has recesses on the outer surface of the head. The screw shank is contained in a sleeve with additional gripping sections to facilitate placement of the screw.

### SUMMARY OF THE INVENTION

The present invention can provide a bone treatment device, which allows for reinforced security at the bone interface and/or for delivery of a bone treatment material into a bone portion. The bone treatment device can comprise a shaft member having first and second end portions. A delivery channel can be formed in the shaft member to assist administering a bone treatment material to a predetermined bone portion or tissue. The delivery channel can comprise a chamber formed in the shaft member and extending from the first end portion toward the second end portion. The chamber so formed is capable of receiving and guiding the bone treatment material toward the predetermined bone portion or tissue. The delivery channel can also comprise one or more port members formed on the shaft member and communicating with the chamber. The port members are capable of delivering the bone treatment material to the predetermined bone portion or tissue for bone treatment.

When the bone treatment device is inserted and/or implanted into a bone portion, such as osteoporotic, or cancellous bone, or periosteum, a bone treatment material can be introduced into the bone treatment device. For example, the treatment material can be injected into the device from the first open end portion and guided by the chamber to the port members for delivery to the bone portion or tissue. The bone treatment device can be used to treat various kinds of trauma, including spine, femur head, humerus head, short bone, and long bone, with or without osteoporosis.

According to one aspect of the present invention, the bone treatment device can be a bone fixation device, such as a bone screw. By delivering an injectable bone treatment material, such as a bone cement or other biomaterials, through the material delivery channel, the injected cement can flow out of the device and into the cancellous bone via the port members. After setting of the bone cement, the cement can provide additional anchorage to reinforce the bone fixation device purchase at the bone interface. Additionally or alternatively, the bone treatment device can be a treatment delivery device for delivering medications, or other materials into a bone portion or tissue via the material delivery channel.

According to another aspect of the present invention, a bone treatment method can be provided, which can use the bone treatment device as described above. For example, the bone treatment device can be inserted and/or implanted in a bone portion. A bone treatment material can be administered into the bone treatment device. The bone treatment material can be guided through the chamber and delivered into the bone portion or tissue via the port members.

### BRIEF DESCRIPTION OF THE DRAWINGS

The detailed description of the present invention will be better understood in conjunction with the accompanying drawings. Nevertheless, the accompanying drawings are for illustrative purposes only; the present invention is not limited to the exemplary embodiments shown in such drawings.

FIG. 1 shows an exemplary bone treatment device being inserted and/or fixed onto a bone portion.

FIG. 2 shows a first exemplary embodiment of the bone treatment device.

FIG. 3 shows a second exemplary embodiment of the bone treatment device.

FIG. 4 shows a third exemplary embodiment of the bone treatment device.

FIG. 5 is a longitudinal cross-section view of the bone treatment device as shown in FIG. 3.

### DETAILED DESCRIPTION OF THE INVENTION

Exemplary bone treatment devices and methods embodying the principles of the present invention are shown throughout the drawings and will now be described in detail. In the following description of various embodiments of bone treatment devices and methods, similar elements or components thereof are designated with reference numbers; redundant description is omitted.

The present invention can provide a bone treatment device 1 capable of treating various bone conditions, such as osteoporotic, cancellous, or other bone portions. The bone treatment device 1 can facilitate fixing bone fractures and/or delivering a bone treatment material to a bone portion or tissue for bone treatment. In particular, the treatment device 1 is capable of providing a reinforced bonding at the bone interface.

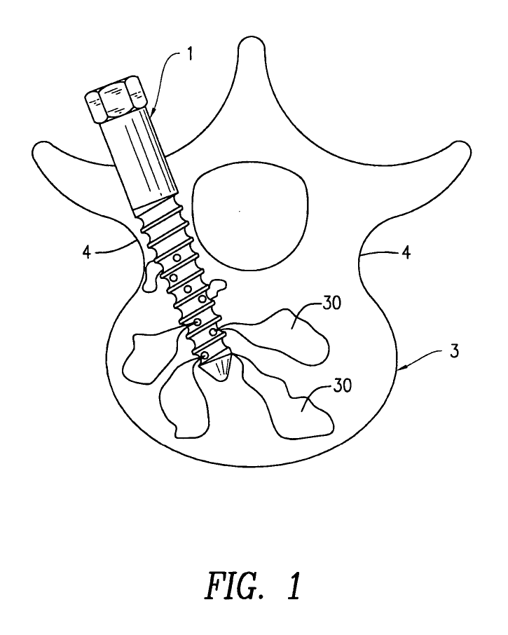

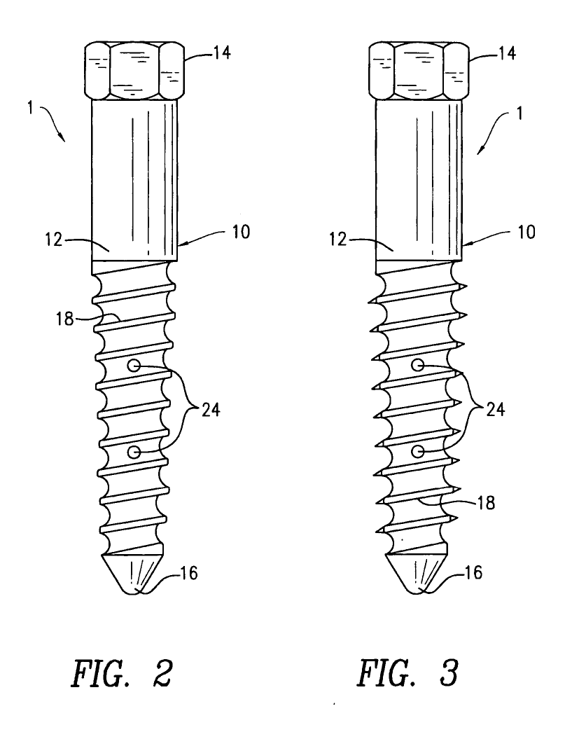

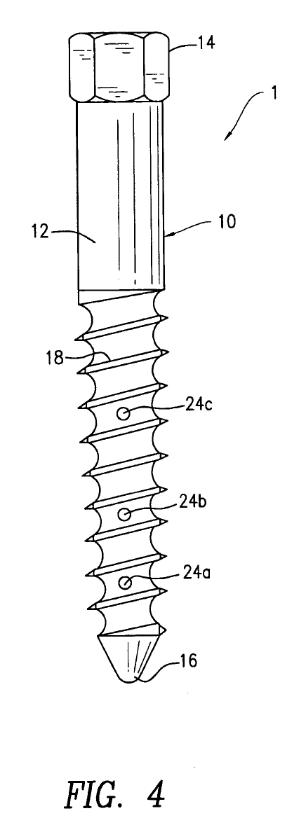

The bone treatment device 1 can be in various forms, such as bone screws, rods, pins, bars, plates, implants, or the like. For example, the bone treatment device 1 can be in the form of a bone screw. In an exemplary embodiment, such as shown in FIGS. 1 and 2, the bone screw 1 can be of a non-self-tapping type. In another exemplary embodiment, such as shown in FIGS. 3 and 4, the bone screw 1 can be of a self-tapping type. It will be appreciated that other types of bone screw and/or other forms of the bone treatment device 1 are also within the scope of the present invention.

In one embodiment, the bone treatment device 1 can comprise a shaft member 10 having an outer circumferential surface 12 and first and second end portions 14 and 16. The shaft member 12 can be in various shapes. In an exemplary embodiment, the shaft member 10 can elongated. In another exemplary embodiment, the shaft member 10 can have various cross-sectional shapes, such as circular, square, and rectangular shapes.

In one exemplary embodiment, the bone treatment device 1 can be a bone screw, of which the shaft member 10 can have a threading member 18 formed on a portion of the circumferential surface 12. In an exemplary embodiment, the threading member 18 can be formed on the second end portion 16. In another exemplary embodiment, the threading member 18 can be formed on the second end portion 16 and extend to middle section of the shaft member 10. In a further exemplary embodiment, the shaft member 10 can comprise a screw head member. It will be appreciated that other forms of the shaft member 10 are also within the scope of the present invention.

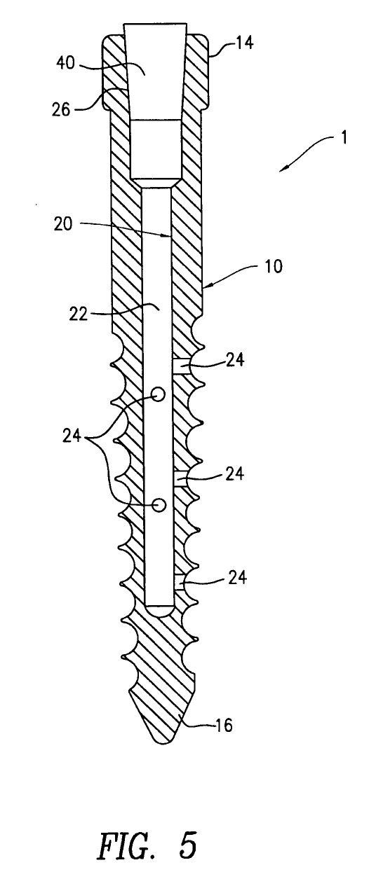

In another embodiment, the bone treatment device 1 can comprise a material delivery channel 20 at least partially defined in the shaft member 10. The material delivery channel 20 so formed is capable of delivering a bone treatment material 30 to a bone portion or tissue for bone treatment. In one exemplary embodiment, such as shown in FIG. 5, a chamber 22 can be formed in the shaft member 10 and extending from the first open end portion 14 toward the second end portion 16. In an exemplary embodiment, the chamber 22 can extend through the first end portion 14 for receiving a bone treatment material 30 as will be described below. In another exemplary embodiment, one or more port members 24 can be formed on the shaft member communicating with the chamber 22. The port members 24 are capable of delivering a bone treatment material 30 to a predetermined bone portion 3 or tissue. In one exemplary embodiment, the chamber 22 and the port members 24 can jointly form the material delivery channel 20. It will be appreciated that other forms of the material delivery channel 20 are also within the scope of the present invention.

The port members 24 can be arranged in various patterns. For example, a plurality of port members 24 can be formed on the shaft member 10 communicating with the chamber 22. In an exemplary embodiment, at least some of the port members 24 can be in a collinear alignment. In another exemplary embodiment, the port members 24 can form a spiral pattern along the shaft member 10. In a further exemplary embodiment, the port members 24 can be spaced from each other. For example, the port members 24 can be spaced from each other in a circumferential direction of the shaft member 10. For example, two adjacent port members 24 can be spaced from each other for about 30° to about 180° in the circumferential direction. In an exemplary embodiment, adjacent port members 24 can be spaced from each other for about 90° in the circumferential direction.

Additionally or alternatively, the port members 24 can be spaced from each other in a longitudinal direction of the shaft member 10. In an exemplary embodiment, two adjacent port members 24 can be spaced from each other in the longitudinal direction for about 1 mm or more. In another exemplary embodiment, two adjacent port members 24 can be spaced from each other in the longitudinal direction for less than about 20 mm. For example, the longitudinal spacing between two adjacent port members 24 can range from about 1 mm to about 20 mm. In one exemplary embodiment, the longitudinal spacing between two adjacent port members 24 can range from about 1.5 mm to about 10 mm. In another exemplary embodiment, two adjacent port members 24 can be spaced from each other in the longitudinal direction for about 3.0 mm. In a further exemplary embodiment, two adjacent port members 24 can be spaced from each other in the longitudinal direction for about 5.0 mm. In a still further exemplary embodiment, such as shown in FIG. 4, adjacent port members 24a and 24b can have different spacing from adjacent port members 24b and 24c in the longitudinal direction. It will be appreciated that other patterns and/or arrangements of the port members 24 are also within the scope of the present invention.

The bone treatment device 1 can be made of various materials, such as stainless steel, titanium, nickel titanium alloy, or other biocompatible materials. In an exemplary embodiment, the device 1 can be made of a material with sufficient strength to support a bone portion. It will be appreciated that various other materials for making the bone treatment device 1 are also within the scope of the present invention.

Optionally, a sealing member 40 can be provided to selectively seal the first end portion 14 of the shaft member 10. For example, the sealing member 40 can be in the form of a plug for fitting into an opening 26 formed at the first end portion 14. In an exemplary embodiment, the sealing member 40 can provide a water-tight sealing for the first end portion 14. For example, the sealing member 40 can have a tapered outer surface to match with a tapered opening 26 formed in the first end portion 14. In another exemplary embodiment, the sealing member 40 can be made of a polyethylene material. It will be appreciated that various other forms or materials for the sealing member 40 are also within the scope of the present invention.

The bone treatment device 1 can be inserted into and/or fixed onto a bone portion or tissue by various conventional methods of bone screw insertion. In an exemplary embodiment, the bone treatment device 1 can be inserted into a predrilled passage formed in the bone portion. For example, when a non-self-tapping bone screw 10 is used, such as shown in FIGS. 1 and 2, a predrilled hole can be provided which can be slightly smaller than the diameter of the bone screw 10 to facilitate the fixation. In another exemplary embodiment, a self-tapping bone screw 10, such as shown in FIGS. 3 and 4, can be directly inserted into the bone portion without a predrilled hole. If desired, additional fasteners can be provided to assist in securing the bone treatment device 1 onto a bone portion. It will be appreciated that other methods for inserting and/or fixing the bone treatment device 1 are also within the scope of the present invention.

In one exemplary embodiment, after the bone treatment device 1 is inserted and/or implanted into a bone portion, a bone treatment material 30 can be introduced into the bone treatment device 1. The bone treatment material 30 can be various biomaterials, medications, drugs, bone cement, bone graft materials, or the like. In an exemplary embodiment, the bone treatment material 30 can be injected into the material delivery channel 20 through the first open end portion 14. The treatment material 30 can be guided by the chamber 22 to the port members 24 for delivery into the bone portion or tissue. It will be appreciated that other methods for administering or delivering the bone treatment material 30 are also within the scope of the present invention.

The bone treatment device 1 can be used for various bone treatment purposes. In one exemplary embodiment, the bone treatment device 1 can be used to fix various bone portions. For example, the bone treatment device 1 can be a bone fixation device, which is adapted to be implanted into the bone portion for bone fixation. In an exemplary embodiment, the bone treatment device 1 can be formed as a pedicle screw 1 and inserted into an osteoporotic vertebral body.

In another exemplary embodiment, the bone treatment device 1 can employ a bone treatment material 30 to provide a reinforced bone fixation. For example, a bone cement 30 can be used to be administered to the osteoporotic vertebral body via the delivering channel 20 in the bone treatment device 1. In an exemplary embodiment, the bone cement 30 can be injected into the bone treatment device 1 and guided by the chamber 22 to the port members 24 for delivery into the cancellous and/or osteoporotic bone. After the cement 30 is set, it is capable of enhancing the secure purchase of the bone/screw interface. In another exemplary embodiment, the bone cement 30 can enter into the bone portion under a sufficient pressure to fill the bone to a predetermined degree for anchoring the bone treatment device 1.

Additionally or alternatively, the bone treatment device 1 can be used as a material delivery device for administering a bone treatment material 30 to a bone portion or tissue. For example, the bone treatment device 1 can deliver a bone treatment material 30, such as a medication or drug, to a bone tissue via the material delivering channel 20. In an exemplary embodiment, the bone treatment material 30 can be injected into the material delivering channel 20 through the first open end portion 14 after the bone treatment device 1 is inserted into the bone tissue. In another exemplary embodiment, the bone treatment material 30 can be present and capped in the bone treatment device 1 by a removable sealing member 40 fit inside the first open end portion 14 at the time of insertion. In a further exemplary embodiment, the medication 30 can be delivered by diffusion or other means over time to the bone tissue for treatment. It will be appreciated that various other applications of the bone treatment device 1 are also within the scope of the present invention.

Various exemplary bone treatment devices 1 will now be described in conjunction with the accompany drawings.

FIG. 1 shows an exemplary bone treatment device 1 being fixed onto vertebra, such as at osteoporotic vertebra, for bone treatment. For example, the bone treatment device 1 can be in the form of a bone screw 1 and fixed to the vertebral body 3, via the pedicle 4. It will be appreciated that various other bone portions and/or tissues can also be treated by the bone treatment device 1, which are also within the scope of the present invention.

After the bone treatment device 1 is inserted and/or implanted in a bone portion, a bone treatment material 30 can be administered by any suitable method. For example, the treatment material 30 can be administered by injection through the material delivery system 20 of the bone treatment device 1 and into the vertebral body 3. Various bone treatment materials 30, such as biomaterials, medications, drugs, bone cement, bone graft materials, or other materials, can be used for various types of bone treatment. In an exemplary embodiment, a bone treatment material 30 is employed to enhance the secure purchase of the bone screw 1 to the bone, such as a vertebral body 3. In another exemplary embodiment, a bone cement 30 can be used to fill up the whole or a portion of the vertebral body 3. After the bone cement 30 is set, the cement 30 can act as an additional anchor to secure the bone treatment device 1.

FIGS. 2 to 5 show various exemplary bone treatment devices 1, which can be formed in various sizes and/or shapes, such as depending on the bone portion to which the device 1 will be applied. For example, the bone treatment device 1 can have various lengths. In an exemplary embodiment, the total length of the bone treatment device 1 can be about 20 mm or more. In another exemplary embodiment, the total length of the bone treatment device 1 can be no more than about 330 mm. For example, the bone treatment device 1 can have a length of from about 20 mm to about 330 mm. In anther exemplary embodiment, the total length of the bone treatment device 1 can be about 35 mm or more. In a further exemplary embodiment, the total length of the bone treatment device 1 can be less than about 130 mm. Preferably, the bone treatment device 1 can have a length from about 35 mm to about 130 mm.

Additionally or alternatively, the bone treatment device 1 can have various cross-section shapes, such as circular, square, and rectangular shapes. For example, the bone treatment device 1 can have a circular cross-section. In an exemplary embodiment, the shaft member 10 can have a diameter of about 2 mm or more. In another exemplary embodiment, the shaft member 10 can have a diameter from about 2 mm to about 10 mm. Preferably, the shaft member 10 can have a diameter from about 3 mm to about 9 mm.

In another exemplary embodiment, the bone treatment device 1 can have a threading member 18 provided on the shaft member 10. For example, the treading member 18 can extend from the second end portion 16 toward the first end portion 14. In an exemplary embodiment, the treading member 18 extends to a middle section of the shaft member 10. The length of the threading member 18 is preferably from about 20 mm to about 80 mm. Additionally or alternatively, the pitch of the threading member 18 can vary. For example, the pitch can be about 1 mm or more. In an exemplary embodiment, the pitch can range from about 1.5 mm to about 5 mm.

In a further exemplary embodiment, the bone treatment device 1 can be provided with a nut member, such as a hexagonal nut. The height of the nut member is preferably from about 5 mm to about 30 mm. Additionally and alternatively, the nut can have a width of from about 3 mm to about 9 mm. It will be appreciated that other structures, shapes, and dimensions of the shaft member 10 and the bone treatment device 1 are also within the scope of the present invention.

FIG. 5 shows an exemplary material delivery channel 20 of the bone treatment device 1. In one exemplary embodiment, the deliver channel 20 can be formed by a chamber 22 and one or more port members 24. The chamber 22 can be formed in the shaft member 10 extending from the first end portion 14 toward the second end portion 16. In an exemplary embodiment, the first end portion 14 can be open to communicate with the chamber 22. Additionally or alternatively, the second end portion 16 of the shaft member 10 can be closed or otherwise sealed.

In one exemplary embodiment, the total length of the chamber 22 can vary. In an exemplary embodiment, the chamber 22 can have a length of at least about 10 mm. In another exemplary embodiment, the chamber 22 can have a length up to about 320 mm. For example, the total length of the chamber 22 can range between about 30 mm and about 320 mm. Preferably, the chamber 22 can have a total length ranging from about 30 mm to about 120 mm.

Additionally or alternatively, the chamber 22 can have various cross-sectional shapes, such as circular, square, rectangular shapes. For example, the chamber 22 can have a circular cross-section. In an exemplary embodiment, the chamber 22 can have a diameter of at least about 1 mm and/or of no more than about 7 mm. For example, the chamber 22 can have a diameter in the range of about 2 mm to about 7 mm. In another exemplary embodiment, the diameter of the opening 26 can be at least about 2 mm and/or no more than about 7 mm. It will be appreciated that other shapes and sizes of the chamber 22 are also within the scope of the present invention.

The port members 24 can be formed on the shaft member 10 to communicate the inner chamber 22 with the outside of the bone treatment device 1. In an exemplary embodiment, the port members 24 can be positioned away from the end portions 14 and 16. The port members 24 can allow for fluid communication and delivery of a bone treatment material 30 from the chamber 22 to a bone portion or tissue to be treated.

The number of port members 24 can be determined, such as the bone portion or tissue or the condition of the bone portion or tissue to which the bone treatment device 1 will be applied. Preferably, the bone treatment device 1 can contain at least two port members 24. In an exemplary embodiment, such as shown in FIG. 5, about ten port members 24 can be provided on the shaft member 10.

In another exemplary embodiment, the port members 24 can be arranged in various patterns or in a random distribution. Preferably, at least some port members are arranged in collinear alignment. In an exemplary embodiment, as shown in FIG. 5, the port members can be evenly spaced in the longitudinal direction of the shaft member 10. In another exemplary embodiment, as shown in FIG. 4, adjacent port members can have different spacing in the longitudinal direction of the shaft member 10. For example, the longitudinal spacing between port members 24a and 24b can differ from that of port members 24b and 24c.

Additionally or alternatively, the port members 24 can have various shapes and sizes. For example, the port members 24 can be narrow slits or holes. In an exemplary embodiment, the port members 24 can have a diameter of about 0.5 mm or more. In another exemplary embodiment, the diameter of the port members 24 can be about 0.5 mm to about 4 mm. Preferably, the port members 24 can have a diameter from about 1 mm to about 4 mm. It will be appreciated that other patterns, shapes, and sizes of the port members 24 are also within the scope of the present invention.

The bone treatment device 1 can be varied to facilitate the various usage. In an exemplary embodiment, the first open end portion 14 can be fitted with a coating, seal, or cap by a suitable material, such as polyethylene, which can prevent the bone treatment material 30 from leaking outside the inner chamber 22. In another exemplary embodiment, the first end portion 14 can be so formed to facilitate the receipt of the sealing member 40. For example, the first end portion 14 can comprise a tapered opening 26, such as shown in FIG. 5, or an inner screw portion (not shown). Additionally or alternatively, the second end portion 16 of the shaft member 10 can be a pointed structure to facilitate the insertion of the bone treatment device 1.

The bone treatment device 1 can be used with various other devices to enhance the secure purchase of the bone treatment device 1 to the bone portion, such as an osteoporotic bone. For example, the bone treatment device 1 can be linked-up with various structural elements, such as plates, rods, pins, bone implants, or the like.

It will be appreciated that the various features described herein may be used singly or in any combination thereof. Therefore, the present invention is not limited to only the embodiments specifically described herein. While the foregoing description and drawings represent a preferred embodiment of the present invention, it will be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiment is therefore to be considered in all respects as illustrative and not restrictive, the scope of the invention being indicated by the appended claims, and not limited to the foregoing description.

1. A bone screw device comprising:
a shaft member having first and second end portions and an outer surface; and
a threaded portion formed on a portion of the outer surface;
wherein the shaft member is provided with an inner chamber and a port member communicating between the inner chamber and the circumferential surface of the shaft member; and
wherein the inner chamber extends from the first end portion and longitudinally through at least a portion of the shaft member.

2. The bone screw device of claim 1, wherein the port member contains a plurality of holes having a diameter of from about 0.5 mm to about 4 mm.

3. The bone screw device of claim 2, wherein at least some of the holes are in collinear alignment.

4. The bone screw device of claim 2, wherein the holes are spaced from each other in a longitudinal direction of the shaft member for about 1 mm to about 20 mm.

5. The bone screw device of claim 1, wherein the shaft member has a length of from about 20 mm to about 330 mm and an outer diameter of from about 2 mm to about 10 mm.

6. The bone screw device of claim 1, wherein the inner chamber has a length from about 10 mm to about 320 mm and a diameter from about 2 to about 7 mm.

7. The bone screw of claim 1, further comprising a sealing member for the inner chamber.

8. The bone screw device of claim 7, wherein sealing member comprises polyethylene.

9. The bone screw device of claim 1 being an implantable screw.

10. A bone treatment device, comprising:
a shaft member having first and second end portions, of which the first end portion is open;
a chamber formed in the shaft member and extending from the first open end portion toward the second end portion; and
one or more port members formed on the shaft member and communicating with the chamber;
whereby after the bone treatment device is implanted into a bone portion, a bone treatment material can be introduced into the chamber from the first open end portion and guided by the chamber to the port members for delivery into the bone portion.

11. The bone treatment device of claim 10, wherein a plurality of port members are formed on the shaft member communicating with the chamber.

12. The bone treatment device of claim 11, wherein the port members are spaced from each other.

13. The bone treatment device of claim 11, wherein the port members are spaced from each other in a longitudinal direction of the shaft member for about 1.5 mm to about 5 mm.

14. The bone treatment device of claim 11, wherein at least some of the port members are in a collinear alignment.

15. The bone treatment device of claim 11, wherein the port members are spaced from each other in a circumferential direction of the shaft member for about 30 degrees to about 180 degrees.

16. The bone treatment device of claim 15, wherein the port members are spaced from each other for about 90 degrees.

17. The bone treatment device of claim 10, wherein the second end portion of the shaft member is sealed.

18. The bone treatment device of claim 10, further comprising a sealing member for selectively sealing the first open end portion of the shaft member.

19. The bone treatment device of claim 10 being a bone fixation device, which is adapted to be implanted into the bone portion for bone fixation.

20. A bone treatment method comprising:
providing a bone treatment device of claim 10; and
introducing bone treatment material into the device through the first opening end portion;
whereby the bone treatment material is guided through the chamber and delivered into the bone portion via the port members.
